(19) United States
(12) Patent Application Publication
(10) Pub. No.: US 2025/0031996 A1
(43) Pub. Date: Jan. 30, 2025

(54) IMAGE DIAGNOSTIC APPARATUS AND METHOD OF MONITORING PATIENT DURING EXAMINATION

(71) Applicant: FUJIFILM Healthcare Corporation, Kashiwa-shi (JP)

(72) Inventors: Ayaka Ikegawa, Chiba (JP); Yukio Kaneko, Tokyo (JP); Takenori Murase, Chiba (JP)

(21) Appl. No.: 18/761,538

(22) Filed: Jul. 2, 2024

(30) Foreign Application Priority Data
Jul. 27, 2023 (JP) 2023-122816

(51) Int. Cl. A61B 5/11; A61B 5/00; A61B 5/055

(52) U.S. Cl. CPC A61B 5/11; A61B 5/055; A61B 5/7425; A61B 5/743; A61B 5/746

(57) ABSTRACT

A technology is provided for ensuring safety of a subject in advance by estimating whether or not the subject is in a dangerous state based on a body motion obtained from a camera video. An image diagnostic apparatus includes a body motion analysis unit, in which the body motion analysis unit includes a body motion indicator calculation section that calculates a quantity related to a body motion of a subject by using a video from an imaging device and calculates an indicator of the body motion by using the quantity and a coefficient determined in advance based on an examination condition, and a patient state estimation section that estimates a state of the subject by using the indicator of the body motion calculated by the body motion indicator calculation section, and alert information is output to the console based on an estimation result of the patient state estimation section.

### CROSS REFERENCE TO RELATED APPLICATION

The present application claims priority from Japanese Patent Application No. 2023-122816, filed on Jul. 27, 2023, the content of which is hereby incorporated by reference into this application.

### BACKGROUND OF THE INVENTION

### 1. Field of the Invention

The present invention relates to an image diagnostic apparatus, and more particularly, to a technology for improving safety of a patient during an examination using the image diagnostic apparatus.

### 2. Description of the Related Art

In an image diagnostic apparatus such as an MRI apparatus or a CT apparatus, a patient (hereinafter, also referred to as a subject) is transported into an examination space in a gantry in which an imaging device is accommodated, and examined in a state of being laid on a patient table. Since an operation room for a doctor or an examination technician who performs an examination to operate the apparatus is separated from the examination space, a state of the subject in the apparatus cannot be understood.

There is a probability that the subject may fall from the patient table or may unexpectedly move due to some causes during the examination, and in such a case, not only may the imaging necessary for image diagnosis be interrupted, but there is also a possibility that the safety of the subject will not be ensured. In order to monitor such a motion of the subject, measures such as attaching a sensor that detects a body motion to the subject have conventionally been taken. However, there is a problem that mounting such a sensor is time-consuming and prolongs the examination time. In response, a method of installing a camera that captures the examination space, recognizing a position of the subject on the patient table from a video of the camera, and issuing a warning in a case where an actual position of the subject deviates from an estimated position, or the like has also been proposed (JP2021-129716A).

### SUMMARY OF THE INVENTION

The technology described in JP2021-129716A is to determine whether the position of the subject has actually moved from the image of the camera, and it is not possible to detect an abnormal state in which danger may occur before the subject moves.

An object of the present invention is to provide a technology capable of ensuring safety of a subject in advance by estimating whether or not the subject is in a dangerous state based on a body motion obtained from a camera video.

In order to achieve the above-described object, according to the present invention, body motion information detected from the camera video and an examination condition affecting the body motion are used to estimate a degree of a dangerous state of the subject, and an alert corresponding to a degree of danger is issued to an operator outside an examination room. As a result, the operator can respond before the subject actually falls into the dangerous state.

That is, according to an aspect of the present invention, there is provided an image diagnostic apparatus comprising: a gantry that provides an examination space; an imaging unit that images a subject disposed in the examination space; a body motion analysis unit that acquires a video from an imaging device which images an inside of the gantry and that analyzes a motion of the subject; a console that is installed at a location different from the gantry and has a user interface function; and a display control unit that controls an output to the console. The body motion analysis unit includes a body motion indicator calculation section that calculates a quantity related to a body motion of the subject by using the video from the imaging device and calculates an indicator of the body motion by using the quantity and a coefficient determined in advance based on an examination condition, and a patient state estimation section that estimates a state of the subject by using the indicator of the body motion calculated by the body motion indicator calculation section, and alert information is output to the console based on an estimation result of the patient state estimation section.

In addition, according to another aspect of the present invention, there is provided a method of monitoring a patient during imaging, the method being for acquiring a video of a subject placed in an examination space, observing a state of the subject, and issuing a necessary alert according to the state of the subject, the method comprising: calculating a quantity related to a body motion of the subject and calculating an indicator of the body motion by using the quantity and a coefficient determined in advance based on an examination condition; and estimating the state of the subject by using the indicator of the body motion. A predetermined alert issuance control is performed according to the estimated state of the subject. Here, an imaging condition is a condition relevant to the motion of the patient, and examples of the imaging condition include a position and a posture of the subject, an examination body part, and the presence or absence of medication administration to the subject.

According to the aspects of the present invention, the state of the subject is estimated by using information on continuous body motions understood from the video of the camera and a condition highly relevant to the motion of the subject during the examination. Then, alert information corresponding to the estimated state is issued. As a result, the operator can preemptively and easily understand a patient state and can prevent a dangerous state caused by a falling accident or a sudden change. In addition, a need to attach the sensor or the like for monitoring the motion of the patient is eliminated, thereby reducing the workload for the operator and the discomfort for the patient.

### BRIEF DESCRIPTION OF THE DRAWINGS

FIG. 1 is a diagram showing an example of an image diagnostic apparatus to which the present invention is applied.

FIG. 2 is a functional block diagram of the image diagnostic apparatus.

FIG. 3 is a diagram showing an operation of Embodiment 1 of the image diagnostic apparatus.

FIG. 4 is a diagram illustrating a flow of processing of a body motion analysis unit of Embodiment 2.

FIG. 5 is a diagram showing an example of a table used by the body motion analysis unit.

FIG. 6 is a diagram showing another example of the table used by the body motion analysis unit.

FIGS. 7A and 7B are diagrams showing a monitor screen during an examination.

FIG. 8 is a diagram showing a flow of processing of the body motion analysis unit of Embodiment 3.

FIG. 9 is a diagram showing an example of a monitor screen provided in a gantry.

FIG. 10 is a diagram showing an example of a monitor screen of a console.

### DESCRIPTION OF THE PREFERRED EMBODIMENTS

Hereinafter, embodiments of an image diagnostic apparatus according to the present invention will be described with reference to the drawings.

### Embodiment 1

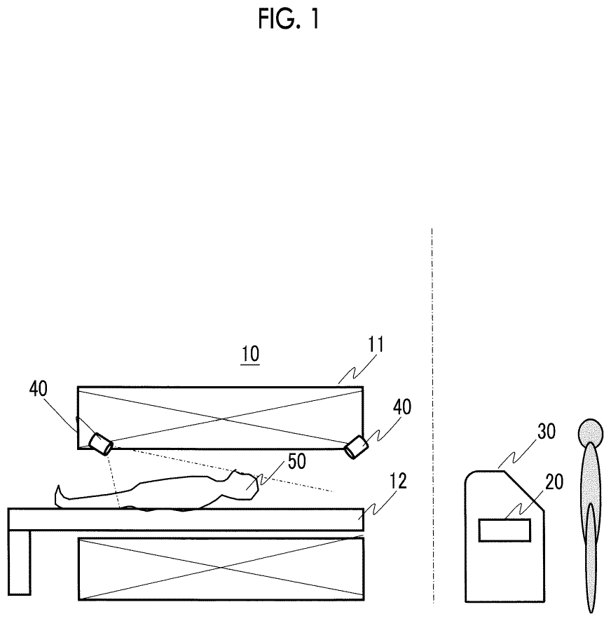

FIG. 1 is a diagram showing an outline of an image diagnostic apparatus 1 of the present embodiment, and as shown in the drawing, the image diagnostic apparatus 1 includes, in broad terms, an imaging unit 10, a computer 20 that controls the imaging unit 10 and that processes a signal measured by the imaging unit 10 to generate an image, and a console 30 that is connected to the computer 20 and is operated by an operator. Although not shown in FIG. 1, the console 30 comprises an input device, such as a keyboard or a mouse, and a display device (monitor), and has a function as a user interface unit (UI unit 300).

Usually, the computer 20 and the console 30 are installed in an operation room different from an examination room in which the imaging unit 10 is placed, and the operator disposes a subject in a predetermined examination space of the imaging unit 10, and then controls the operation of the imaging unit 10 while operating the console 30 in the operation room to perform an examination.

The imaging unit 10 has different configurations depending on the modality, but for example, in a case of an MRI apparatus, a static magnetic field magnet that generates a static magnetic field, a gradient magnetic field coil that generates a gradient magnetic field, an RF transmission coil and a transmitter that irradiates the subject with a high-frequency magnetic field, a receiver to which an RF receive coil that receives a nuclear magnetic resonance signal generated from the subject is connected, and the like are provided, and these are accommodated in a gantry 11 that provides the examination space.

The gantry 11 varies depending on a direction of the static magnetic field generated by the static magnetic field magnet, but in a horizontal magnetic field type MRI apparatus, the gantry has a cylindrical gantry shape with a cylindrical examination space formed inside. A subject 50 is transported into the examination space and is examined in a state of being laid on a patient table 12.

The imaging unit 10 also varies depending on a CT apparatus or a PET apparatus, but the gantry shape is usually cylindrical, and the subject is transported to the examination space inside the gantry. However, the present invention is not limited to the cylindrical gantry and is applicable, for example, to a case where the magnet is disposed above and below the examination space.

A camera (imaging device) 40 that monitors the subject during movement of the subject between the examination space and the outside or during the examination is attached to the gantry 11. It is preferable that the camera 40 is a camera that covers a wide range or a movement range of the subject, such as a wide-angle camera or a tilt camera. An attachment position of the camera and the number of the cameras are not particularly limited, but for example, one or two cameras are installed in the vicinity of openings on both sides of the gantry 11.

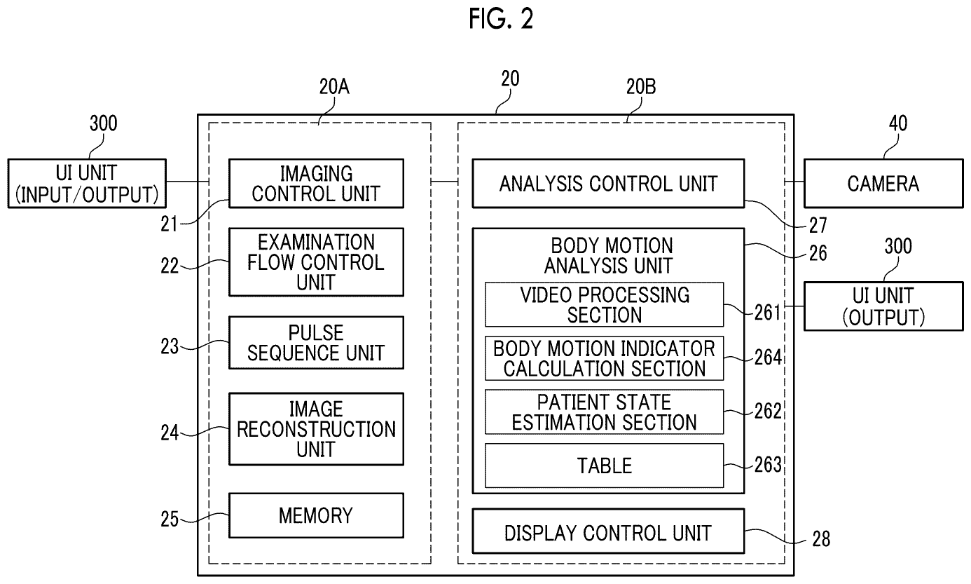

The camera 40 has a function of transmitting the captured video to the image diagnostic apparatus 1 (computer 20), and the computer 20 performs state estimation of the patient by using the video from the camera 40. FIG. 2 shows a configuration example of the computer 20 for implementing this function. Here, as an example, a configuration example in a case where the image diagnostic apparatus is an MRI apparatus will be described.

In the shown example, the computer 20 is shown by being divided into a processing unit 20A that performs processing as a control and calculation system of a normal image diagnostic apparatus and a processing unit 20B that performs processing related to body motion analysis, but these may be constructed in a single computer or may be separate computers.

The processing unit 20A comprises an imaging control unit 21 that controls the operation of the imaging unit, an examination flow control unit 22 that controls an examination flow composed of a plurality of imaging operations, a pulse sequence unit 23, an image reconstruction unit 24, and a memory 25, and is connected to the UI unit 300 provided in the console 30. The functions of these individual units are the same as those of the conventional MRI apparatus, and the pulse sequence unit 23 calculates, for each imaging operation included in the examination flow, a pulse sequence to be used for imaging by using a predetermined pulse sequence selected from among a plurality of pulse sequences and imaging parameters thereof. The imaging control unit 21 controls the imaging unit 10 such that each element constituting the imaging unit 10 operates in accordance with the calculated pulse sequence. The examination flow control unit performs a control such that the imaging is performed in accordance with an examination flow set in advance. The image reconstruction unit 24 performs image reconstruction or other image processing by using measurement data consisting of the nuclear magnetic resonance signals collected by the receiver by executing the imaging. The memory 25 stores information necessary for these individual units to operate, for example, an examination flow, a pulse sequence, and the like set in advance.

The processing unit 20B is a unit that performs the body motion analysis, and receives a video signal from the camera 40, performs the body motion analysis, and performs necessary display, such as an alert, on a monitor of the UI unit 300. In order to implement these functions, the processing unit 20B comprises a body motion analysis unit 26, an analysis control unit 27 that controls an operation of the body motion analysis unit 26, and a display control unit 28 that displays an output as an analysis result of the body motion analysis unit 26 on the display device. In addition, the body motion analysis unit 26 comprises a video processing section 261 that processes the video signal from the camera, a patient state estimation section 262 that estimates a patient state by using the information processed by the video processing section 261, and a memory that stores data or a table 263 necessary for the calculation of the video processing section 261 or the patient state estimation section 262. Further, the patient state estimation section 262 includes a function of a body motion indicator calculation section 264 that calculates a body motion indicator, which will be described below. The memory 25 of the unit 20A may also serve as the function of the memory of the body motion analysis unit 26.

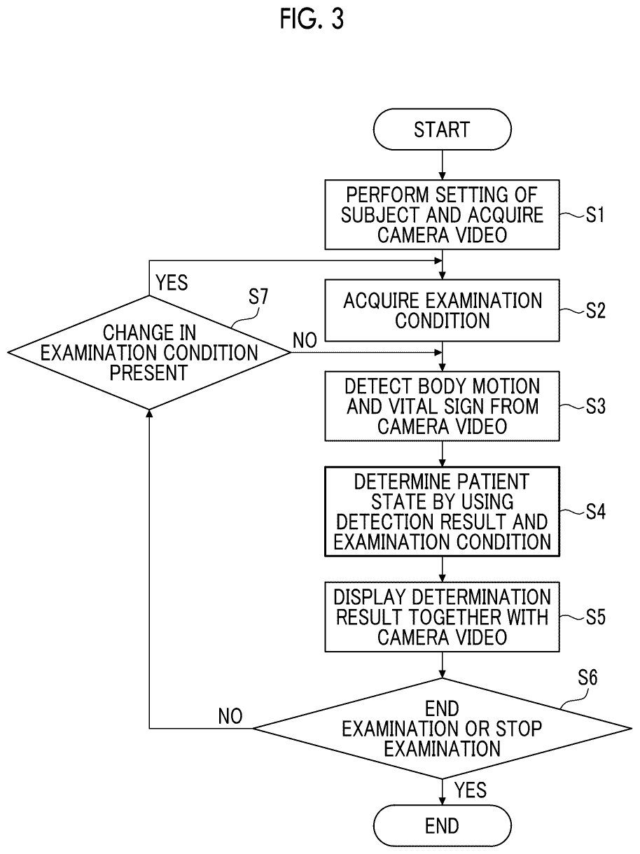

Next, an outline of processing of the image diagnostic apparatus having the above-described configuration will be described with reference to FIG. 3.

First, the subject 50 is laid on the patient table 12, is disposed in the examination space in the gantry (setting of the subject), and the acquisition of the camera video is started (S1). The acquisition of the camera video may be started before the subject 50 is transported to the examination space. In this case, the analysis control unit 27 imports an examination condition into the body motion analysis unit 26 (S2). The examination condition includes an examination body part, a subject posture during imaging (a supine position, a prone position, a lateral position, and the like), a transport direction into the examination space (head-first transport: HF and feet-first transport: FF), the presence or absence of sedation by anesthesia, and the like. The analysis control unit 27 imports these examination conditions via an input from the imaging unit 10 or the console 30. Some of the examination conditions can also be obtained from the camera video.

The video processing section 261 detects vital signs of the subject 50, such as the body motion and the respiration, from the camera video sent from the camera 40 (S3). The detection of the body motion or the respiratory motion is performed as follows, for example.

First, the video from the camera 40 is read, and a motion vector is calculated. The motion vector can be calculated by using, for example, a known computer vision technology such as optical flow, and the motion vector is calculated for each pixel of the image.

Next, the body motion is calculated by using the motion vector. In a case where the camera 40 is a stereo camera, the body motion is obtained as an absolute value because the distance from the camera is obtained, but in a case of a single camera, the body motion is calculated as a relative value. In the calculation of the body motion, a value for each of small regions obtained by dividing the image into a plurality of regions is calculated. In this case, in order to distinguish between respiratory motions in which vertical motions of the abdomen are predominant, and non-periodic body motions (also simply referred to as a body motion) in which the direction of the motion is not regular, different calculation equations are used. Details of the calculation method will be described in the embodiment to be described below.

The patient state estimation section 262 estimates whether or not the patient is in a dangerous state or a degree of the dangerous state from the body motion or the respiratory motion detected by the video processing section 261 (S4). In the estimation of the dangerous state, first, the body motion indicator calculation section 264 multiplies the quantified body motion or respiratory motion by a weight corresponding to the examination condition to calculate an indicator of the body motion, particularly an indicator representing the dangerous state. The patient state estimation section 262 determines the state of the patient by comparing the indicator calculated by the body motion indicator calculation section 264 with a threshold value set in advance.

As described above, the examination conditions for determining the weight include the examination body part, the subject posture during imaging, the transport direction into the examination space, the presence or absence of the sedation, and the like. For example, in a case where the examination body part is a site affected by the respiratory motion, such as the abdomen, a small weight is applied because relatively large motions are expected in a normal state, but in the head or legs, a large weight is applied because motions in the head or legs are more likely to lead to dangerous motions. In addition, in a case where a large body motion is detected in a stable posture such as a supine position or a prone position, a large weight is applied because the body motions are more likely to lead to dangerous motions. Since the administration of sedation by anesthesia is performed in a case where there is a high risk of body movement, such as thrashing, even slight motions are more likely to lead to the dangerous state. Therefore, a large weight is applied.

A weighting value is determined in advance for each of these examination conditions, and the body motion indicator calculation section 264 calculates the indicator by applying the weighting value of the examination condition of the examination currently being performed to the numerical value of the body motion. In a case where there are a plurality of weighting values, the weighting values may be applied as a combined weighting value to calculate the indicator.

In the estimation of the dangerous state using the indicator, it may be determined whether the state is the dangerous state or the calm state by using one threshold value, but a determination may be made by using a plurality of threshold values to divide the degree of the body motion into a plurality of stages. For example, a determination is made as to a calm state, a slight motion, a state requiring imaging stop, a dangerous state that may lead to an accident occurrence, and the like. In addition, a state in which there is a significant positional deviation but not a dangerous state and re-imaging is required may be included in one stage.

The weighting value and the degree (stage) of the dangerous state based on the indicator, which are used for the estimation of the dangerous state by the body motion analysis unit 26 (the body motion indicator calculation section 264 and the patient state estimation section 262), can each be determined in advance and stored in the memory as the table 263, and the body motion analysis unit 26 performs processing by referring to the table in the calculation of the indicator and the above-described determination.

The display control unit 28 presents the determination result of the patient state estimation section 262 on the monitor of the UI unit 300 (S5). The presentation method can employ various aspects, but basically, the determined result is displayed on the monitor of the UI unit 300 in a form of a warning together with the camera video. On this display screen, in addition to the camera video, the vital information obtained by the analysis of the video processing section 261 and the vital information that is the basis for the determination may be displayed together, and an alert using a warning sound or a voice may further be issued in addition to the display on the monitor.

As a result, the user who is at a location away from the subject can immediately understand the change in the body motion of the patient that cannot be determined only from the camera video, particularly the body motion that may lead to the dangerous state, through the console 30, and can take an appropriate response, and it is possible to prevent the fall or other accidents of the subject beforehand.

The above-described processing, S3 to S5, is repeated until the examination ends, except for a case where the examination should be stopped according to the determination result of the patient state (S6). In a case where the examination condition is changed by resuming the examination, or in a case where the examination condition varies depending on the imaging operation in a series of examinations including a plurality of imaging operations (S7), S2 to S5 are repeated.

According to the present embodiment, the body motion of the subject is quantified through the analysis of the camera video, and the indicator obtained by weighting a numerical value related to the body motion with the weight corresponding to the examination condition is calculated, and the degree of danger is determined by using this indicator. As a result, it is possible to eliminate complicated work such as mounting a device for obtaining the vital information on the patient, and it is possible to issue an appropriate warning before the patient actually falls into a dangerous state.

In addition, by tabulating the indicator, the determination result using the indicator, and the like in advance, the processing can be simplified, and the warning can be issued without time delay.

Next, an embodiment of specific processing will be described based on the outline of the above-described embodiment. In the following embodiment, since the configuration of the apparatus is the same as that of Embodiment 1, overlapping descriptions will be omitted, and descriptions will be made with reference to FIG. 2 showing the apparatus configuration as appropriate.

### Embodiment 2

In the present embodiment, the video processing section 261 calculates an amplitude (AMP) and a duration time (Time) of the body motion from the camera video, and a determination is made using an indicator R obtained by multiplying the calculated amplitude and duration time by a weight (w) based on the examination body part, the posture of a person to be examined, and the presence or absence of sedation.

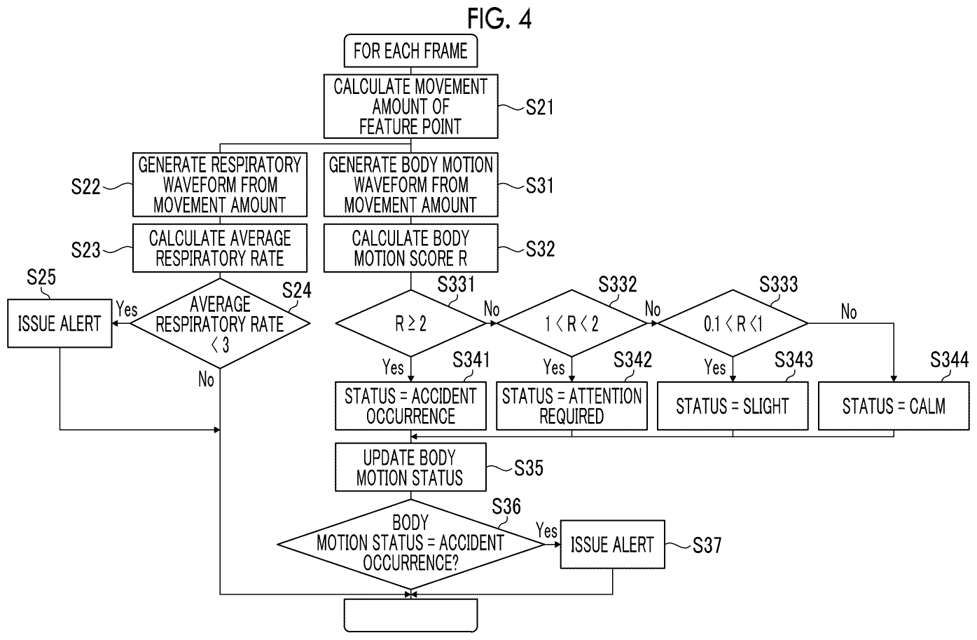

Hereinafter, a flow of processing performed by the body motion analysis unit 26 will be described with reference to FIG. 4. FIG. 4 shows processing for each frame of the video signal sent from the camera 40.

In a case where the video processing section 261 receives the video signal of one frame, the video processing section 261 calculates a change in the subject position by using a difference from the video of a previous frame (S21). Specifically, the optical flow of two frame images is calculated, and a movement amount of a feature point on the subject is calculated. The feature point is a point having a shape-related feature and is, for example, the top of the head, the tip of the chin, both shoulders, the tips of the hands or feet, or the like. Alternatively, a point determined in advance, such as a predetermined position in the mounted receive coil, can be registered as the feature point. In a case of detecting the waveform of the respiratory motion, which will be described below, it is preferable to register a site where the respiratory motion can be detected as the feature point.

The registration of the feature point can be performed by receiving and registering one or a plurality of feature points on the display screen on which the video of the subject is displayed, via the UI unit 300. Alternatively, instead of being registered in advance, a mask for extracting a predetermined region may be created, and the region (monitored region) to be monitored for the body motion may be extracted by using the mask.

For the calculation of the movement amount, a velocity vector representing a change in the pixel position between frames obtained by the optical flow is used to calculate the movement amount as follows. In a case where horizontal and vertical directions of the image are denoted as an x direction and a y direction, respectively, and the velocity in the x direction of a predetermined pixel is denoted as Vx and the velocity in the y direction is denoted as Vy, assuming that the predominant direction of the respiratory motion is mainly a vertical motion with respect to a horizontal plane (patient table surface), and the main direction of the respiratory motion is the y direction, the respiratory motion Vresp is calculated by Equation (1).

$$Vresp = Vy \qquad ( 1 )$$

On the other hand, since the body motion Vbody moves in an irregular direction, the body motion Vbody is calculated by using, for example, Equation (2).

$$V body = Vx \qquad ( 2 )$$

Depending on the body motion direction, positive and negative values can be taken. Therefore, the magnitude thereof is obtained as an absolute value by using, for example, Equation (3).

$$Abody = {( V x )}^{2} \qquad ( 3 )$$

In a case where the main direction of the respiratory motion is different from Vx or Vy depending on the attachment angle of the camera, instead of Equation (1), Equation (4) may be used by using an angle θ of an XY plane with respect to the horizontal plane.

$$V resp = Vx cos θ - Vy sin θ \qquad ( 4 )$$

In addition, for the body motion, in order to minimize the inclusion of the motion of the respiratory motion, the body motion is calculated by using Equations (5) and (6) by taking into consideration the motion in a direction orthogonal to Vresp.

$$Vbody = Vx sin θ + Vy cos θ \qquad ( 5 )$$

$$Abody = {( Vx sin θ )}^{2} + {( Vy cos θ )}^{2} \qquad ( 6 )$$

As described above, in a case where the movement amount is calculated for each of the respiratory motion and the body motion, the video processing section 261 generates a respiratory waveform and a body motion waveform based on the movement amount of the feature point or the monitored region (S22, S31). For the respiratory motion, the movement amount of the feature point for detecting the respiratory motion is generated as the waveform. For the body motion, the movement amount of the feature point (for example, the head, the legs, or both) different from the feature point for the respiratory motion detection is generated as the waveform.

Next, in order to determine the patient state, the patient state estimation section 262 calculates an average respiratory rate from the respiratory waveform (S23). In a case where the average respiratory rate calculated in step S23 is decreased for a certain period as compared with the normal respiratory rate of a person (for example, 3), it is determined to be an abnormal situation, that is, it is determined to be in a state in which an alert should be issued (S24, S25). As a result, a state that may lead to a severe accident of apnea can be monitored. Although a case has been described here in which the respiratory rate is decreased for a certain period based on the respiratory waveform, a state in which the respiratory rate is abnormally increased can also be included in the determination of the abnormal situation.

The patient state estimation section 262 calculates a body motion score (indicator) R from the body motion waveform (S32). The body motion score R is obtained by multiplying the amplitude (Amp) and the duration time (Time) of the body motion by a predetermined weight (W) and can be calculated by using, for example, Equation (7) or (8).

$$R = w ( Am p \times Time ) \qquad ( 7 )$$

$$R = w 1 \cdot Amp + w 2 \cdot Time \qquad ( 8 )$$

Equation (7) represents a case where the product of the amplitude and the duration time is standardized, the value is regarded as a single quantity quantifying the body motion, and the value is multiplied by the weight. Equation (8) is obtained by multiplying the amplitude and the duration time by different weights w1 and w2, respectively. In Equation (8), the sum of the weighted amplitude and the weighted duration time is used as the body motion score R, but the body motion score R may be represented by further applying an additional weight W to the sum. In addition, the indicator representing the body motion is not limited to the body motion score represented by Equation (7) or (8) as long as it is a function of the quantified body motion and the weight. Further, in Equations (7) and (8), a single indicator is calculated by using the amplitude and the duration time, but it is also possible to provide an indicator for each of the =amplitude and the duration time.

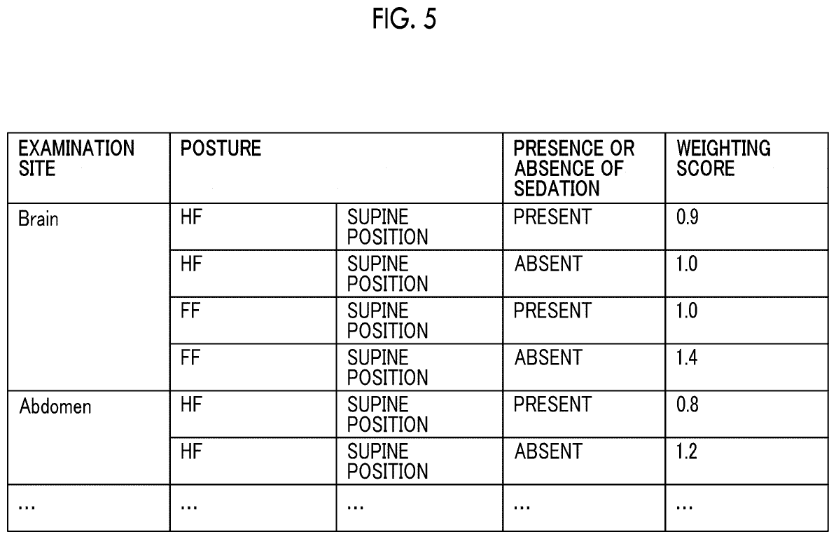

The weight w or the weights w1 and w2 to be used for the calculation of the indicator are stored in the memory in advance by being tabulated in advance as the table 263 for each examination condition. FIG. 5 shows an example of a weight table. In this example, the posture and the presence or absence of sedation are classified for each examination body part, and a predetermined weight is set for each of the classifications.

In addition, for the body motion score R, three threshold values, 0.1, 1, and 2, are set, and four body motion statuses partitioned by these threshold values are determined. Specifically, the patient state estimation section 262 determines the body motion status as follows: an accident occurrence state in a case where R is equal to or more than 2; an attention-required state in a case where R is more than 1 and less than 2; a state in which a slight body motion has occurred in a case where R is more than 0.1 and equal to or less than 1; and a calm state in a case where R is equal to or less than 0.1 (S331 to S333, and S341 to S344). In a case where any of the determination results is obtained, the body motion analysis unit 26 updates the determination result (body motion status) in the previous frame (S35). In a case where the body motion status after the update is “accident occurrence”, the alert is issued (S36 and S37), and the processing of the next frame is started.

The relationship between the body motion score and the body motion status and the control corresponding to the body motion status can be stored in advance in the memory, and the analysis control unit 27 and the display control unit 28 operate based on the control procedure stored in the memory. Specifically, the patient state estimation section 262 determines the body motion status by using the relationship between the body motion score and the body motion status, and the display control unit 28 performs a control such as alert issuance.

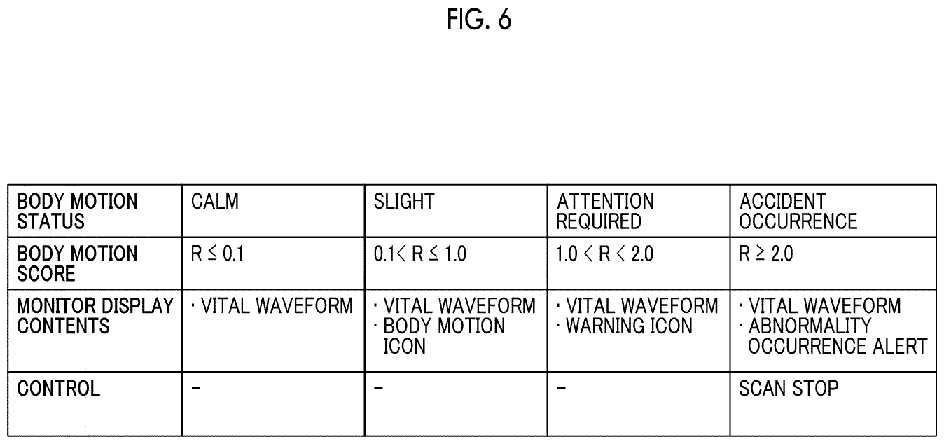

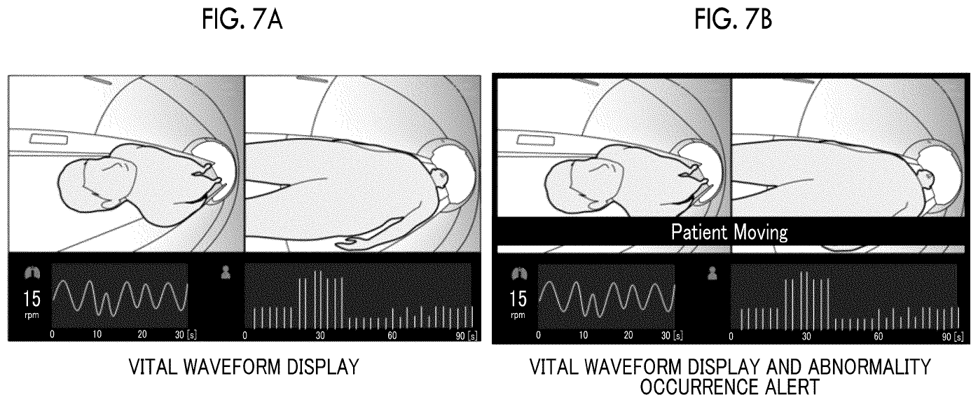

FIG. 6 shows an example of the body motion status determination and the subsequent display control. As shown in the drawing, in a case where the body motion score R is equal to or less than 0.1 and the body motion status is determined to be in the calm state, as shown in FIG. 7A, only the camera video and the vital waveform (the respiratory motion waveform and the body motion waveform) are displayed on the monitor screen of the UI unit. The examples shown in FIGS. 7A and 7B are examples in which the cameras 40 are respectively installed at opening portions on both sides of the gantry 11, and the videos from these two cameras, a video from the head side and a video from the feet side, are displayed, and the respiratory motion waveform (left) and the body motion waveform (right) are displayed below these camera videos.

In other body motion statuses as well, the display control unit 28 continuously displays the camera video and the vital waveform and displays an icon or an alert with different contents on the screen according to the body motion status. For example, a body motion icon indicating that a body motion has occurred is displayed in a case of the slight body motion, and a warning icon is displayed in a case of the attention-required state. In the accident occurrence state or a state significantly close to the accident occurrence state, as shown in FIG. 7B, the alert is displayed in a display mode (red color, blinking, or the like) that attracts the user's attention. In this case, an alert sound is also simultaneously issued. In addition, in a case where the body motion status is determined to be in this state, although not shown in the flowchart of FIG. 4, the information is also passed to the imaging control unit 21. As a result, the imaging control unit 21 performs a control such as stopping the scan. In such a severe accident occurrence, it is also possible to automatically stop the scan or allow the user to select the automatic stop of the scan.

As described above, with the determination algorithm (FIG. 4) of the present embodiment, the abnormality of the respiratory motion and the status of the body motion are monitored in parallel, and in a case where any one of the abnormality of the respiratory motion or the status of the body motion is in a state in which the alert should be issued, the user is immediately notified of the state. As a result, it is possible to detect not only the risk posed by the body motion in which the subject moves but also the respiratory irregularities or the like, and it is possible to further enhance the safety during the examination.

In addition, according to the present embodiment, since the body motion of the subject is divided into a plurality of stages from a body motion that is allowable to a body motion that reaches the dangerous state, and a control corresponding to each stage is performed, the user can take an appropriate response to the body motion, and an unnecessary alert can be reduced. In this case, by determining (tabulating) in advance the relationship between the body motion score and the body motion status, and the relationship between the body motion status and the control corresponding to the body motion status, it is possible to perform the control such as alert issuance very quickly after the occurrence of the body motion, and a rapid response can be made.

Although the present embodiment has been described by focusing on the determination algorithm of FIG. 4, the present embodiment is not limited to FIG. 4, and for example, a waveform from a respiratory motion monitor other than the camera video can also be used for the respiratory motion waveform. In that case, as the function of the body motion analysis unit 26 of the present embodiment, step S22 in FIG. 4 is replaced with a step of reading information (the waveform in a case where the information includes the waveform) from the respiratory motion monitor.

### Embodiment 3

In the Embodiment 1, a case has been mainly described in which the patient state is estimated in a state in which the patient is disposed in the examination space, but an estimation function of the patient state is continued at least from door closing of the examination room through imaging to door opening. During the operation of this estimation function, a control different from the alert during imaging may be performed, while the operator remains in the examination room for setting the subject or the like, or during the movement of the patient table.

For example, since the operator is in the examination room before and after door opening and closing (during the setting of the person to be examined), controls are performed such as reducing the weight of the body motion determination, making the alert less likely to be issued, not notifying the monitor installed in the operation room of the alert in order to prevent erroneous detection of the body motion alert during the patient table movement.

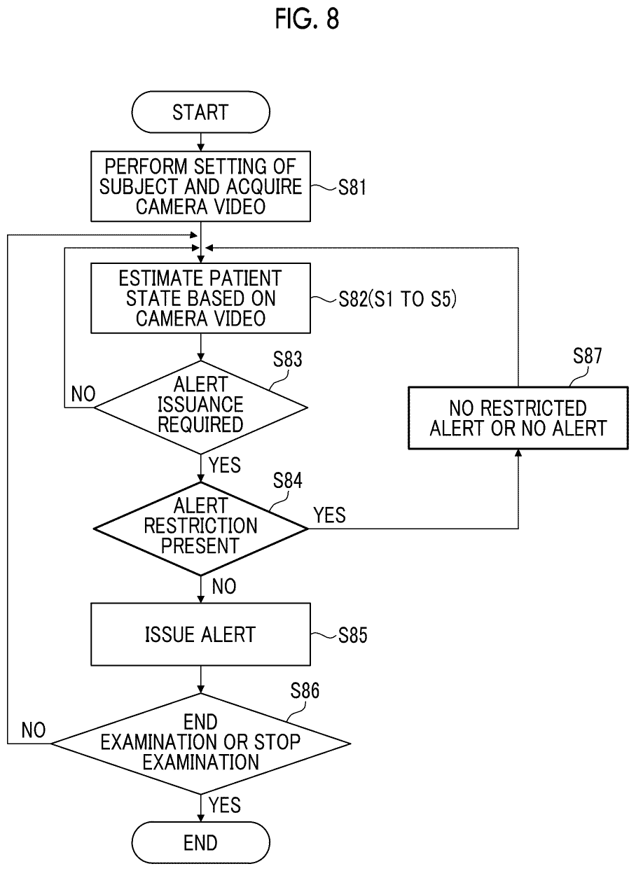

FIG. 8 shows an example of the control flow. The processing of the present embodiment is the same as the processing of Embodiment 1 shown in FIG. 3, but in a case of alert issuance, it is determined whether or not there is a restriction on the alert issuance (S84), and in a case where there is a restriction, processing of issuing a conditional alert in accordance with the restriction or not issuing the alert (S87) is performed.

That is, the acquisition of the camera video is started before the subject 50 is transported into the examination space (S81), and the estimation of the patient state is performed by using the camera video and the examination condition (S82). The processing of step S82 is the same as the processing of steps S2 to S5 in FIG. 3, and in a case where there is no restriction on the alert issuance (S84), processing of the alert issuance is performed according to the patient state (S85), and the process returns to step S82. In the alert issuance, for example, as shown in steps (S331 to S333, S341 to S344, and S35 to S37) of FIG. 4 in Embodiment 2, different alerts are issued according to the value of the body motion score.

On the other hand, in step S84, in a state in which the restriction such as lowering the level of the alert or not issuing the alert is set, the processing is performed in accordance with the restriction (S87). Such a restriction may be automatically switched in conjunction with the movement of the patient table due to the completion of the setting of the subject in the examination space and the end of the imaging, or in a case where the console 30 or the gantry 11 is provided with an operation terminal (gantry monitor), it is also possible for the operator to manually control the alert display, the presence or absence of the warning sound, the detection sensitivity, and the like by operating the operation terminal.

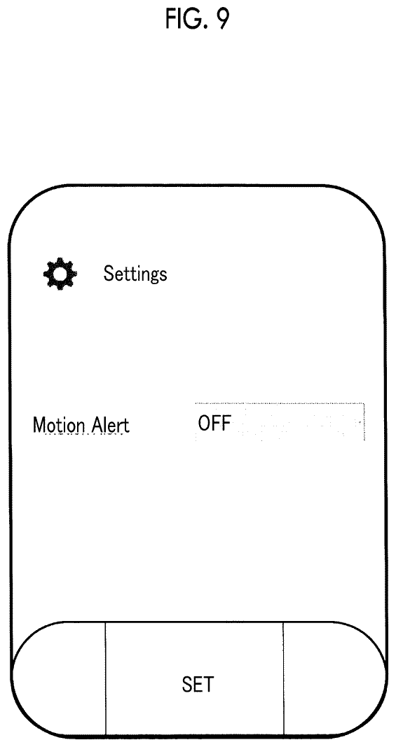

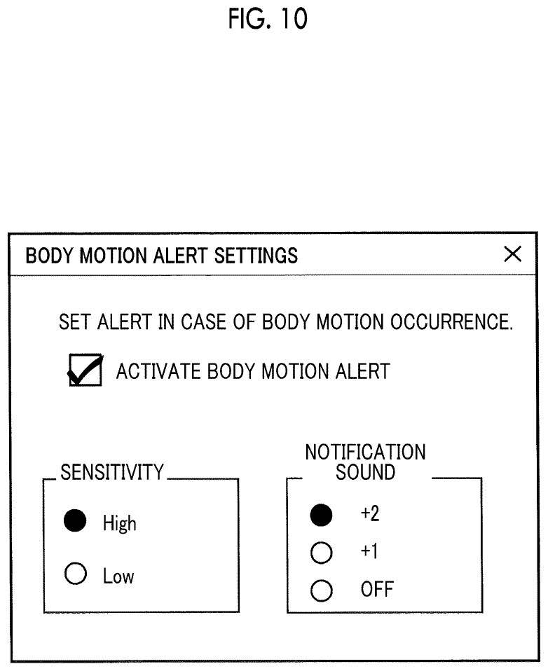

FIG. 9 shows an example of the gantry monitor that receives the operation by the user, and FIG. 10 shows an example of the monitor screen of the console 30. FIG. 9 is an example of a setting screen (setting) by the user, and here, “Motion Alert” (body motion alert) can be set, and in a case where a technician or the like who performs a patient setting or the like in the examination room remains, the alert to the operator is set to a low level, and all the automatic scan and the body motion alert are turned OFF. In this case, in step S84 of FIG. 8, there is a restriction such as not issuing the alert, and the alert is not issued even in a case where the patient moves. After the patient is disposed at a predetermined position in the examination space, the “Motion Alert” is turned ON to enter a state in which a normal alert, that is, an alert without any restrictions, can be issued.

FIG. 10 is an example of the body motion alert setting screen of the console 30, and here, not only ON-OFF of the body motion alert but also the sensitivity or the notification sound volume can be set. The setting of the sensitivity is reflected in the threshold value of the weight of the body motion score in a case of applying the body motion score to the patient state. For example, in a case where the sensitivity is set to be low in a state in which the patient can be visually monitored, the patient state that is usually determined to be “attention required” is determined to be “slight”, and the excessive burden on the operator can be reduced. On the contrary, particularly for a patient or the like in which the body motion that may lead to an accident is anticipated, setting the sensitivity to be high can be more effective in preventing the accident beforehand.

The user setting via the console 30 or the monitor of the gantry 11 can be changed according to the stage of the examination process (during the setting, during the patient table movement, and during scan). Then, the condition related to the alert set via the monitor screen is passed to the body motion analysis unit 26 via the analysis control unit 27 and is reflected in the weight or the threshold value to be used for the estimation of the patient state, and the display control unit 28 performs an alert issuance process in accordance with the condition designated by the user.

According to the present embodiment, the body motion is consistently monitored from the setting of the subject to the end of the examination, and the estimation of the patient state and the alert issuance are controlled according to the process of the examination, so that it is possible to reliably prevent the occurrence of the falling accident or the like while reducing the burden on the operator caused by the excessive alert issuance.

In addition, according to the present embodiment, the configuration is employed in which the user can designate the condition of the body motion alert, so that it is possible to respond flexibly according to the status. In particular, by disposing the monitor that can be operated by the user in both the inside (gantry) of the examination room and the outside (console) of the examination room, the convenience for the user who moves between the inside and the outside of the examination room is improved.

Although the embodiment to which the image diagnostic apparatus of the embodiment of the present invention is applied to the MRI apparatus has been described above, the present invention can also be applied to a modality other than the MRI apparatus, for example, a CT apparatus or a PET apparatus.

In addition, in the embodiment described above, a case has been described in which the dangerous state is estimated by detecting the unintended body motion of the subject, but it is also possible to use the image diagnostic apparatus of the embodiment of the present invention as a function of using the body motion detection function to issue an alert in a case where the subject intentionally causes the body motion, that is, as an operator call, and such a specification is also included in the present invention.

What is claimed is:

1. An image diagnostic apparatus comprising:
a gantry that provides an examination space;
an imaging unit that images a subject disposed in the examination space;
one or more processors configured to acquire a video from an imaging device which images an inside of the gantry and analyze a motion of the subject;
a console that is installed at a location different from the gantry and has a user interface function; and
a display control unit that controls an output to the console,
wherein one or more processors are configured to
calculate a quantity related to a body motion of the subject by using the video from the imaging device and calculate an indicator of the body motion by using the quantity and a coefficient determined in advance based on an examination condition, and
estimate a state of the subject by using the indicator of the body motion calculated by the body motion indicator calculation section, and
the display control unit outputs alert information to the console based on an estimation result of the patient state estimation section.

2. The image diagnostic apparatus according to claim 1,
wherein the examination condition includes any one of a position and a posture of the subject in a case where the subject is inserted into the examination space, an examination body part, or presence or absence of medication pre-administration to the subject.

3. The image diagnostic apparatus according to claim 1,
wherein the one or more processors are configured to calculate an amplitude and a duration time of the body motion of the subject as the quantity related to the body motion and calculate the indicator by using the amplitude and the duration time of the body motion.

4. The image diagnostic apparatus according to claim 3,
wherein the coefficient is a weighting value for the amplitude and the duration time of the body motion.

5. The image diagnostic apparatus according to claim 1,
wherein the one or more processors are configured to hold a table in which a relationship between the indicator of the body motion, the state of the subject, and a control content is set in advance.

6. The image diagnostic apparatus according to claim 5,
wherein the one or more processors are configured to refer to the table to estimate the state of the subject.

7. The image diagnostic apparatus according to claim 5,
wherein the display control unit refers to the table to output the alert information in a case where the control content is alert issuance.

8. The image diagnostic apparatus according to claim 1,
wherein the one or more processors include an analysis control unit that controls analysis of the body motion, and execution and stop of an alert output.

9. The image diagnostic apparatus according to claim 8,
wherein the analysis control unit controls the analysis of the body motion and the stop of the alert output based on a command from a user via the console.

10. The image diagnostic apparatus according to claim 1, further comprising:
a user interface unit that is provided on at least one of the console or an outer surface of the gantry and receives adjustment of the coefficient by a user.

11. The image diagnostic apparatus according to claim 1,
wherein the quantity related to the body motion calculated by the one or more processors includes vital information of the subject, and
the display control unit displays the vital information on a monitor of the console.

12. The image diagnostic apparatus according to claim 1,
wherein the imaging unit is a magnetic resonance imaging apparatus including a magnetic field generation unit that generates a static magnetic field and a gradient magnetic field in the examination space, a transmission unit that irradiates the subject with a high-frequency magnetic field, and a reception unit that receives a nuclear magnetic resonance signal generated from the subject.

13. A method of monitoring a patient during imaging, the method being for acquiring a video of a subject placed in an examination space of an image diagnostic apparatus, observing a state of the subject, and issuing a necessary alert according to the state of the subject, the method comprising:
calculating a quantity related to a body motion of the subject and calculating an indicator of the body motion by using the quantity and a coefficient determined in advance based on an examination condition;
estimating the state of the subject by using the indicator of the body motion; and
performing a predetermined alert issuance control according to the estimated state of the subject.

14. The method of monitoring a patient according to claim 13,
wherein a control of stopping an operation of the image diagnostic apparatus is performed according to the estimated state of the subject.
